(19) United States
(12) Patent Application Publication
(10) Pub. No.: US 2015/0282487 A1
(43) Pub. Date: Oct. 8, 2015

(54) COMPLEX AND USE THEREOF

(71) Applicant: Hong-Ming Lin, New Taipei City (TW)

(72) Inventors: Hong-Ming Lin, New Taipei City (TW); Shuo-Ting Hung, New Taipei City (TW); Wei-Jen Wang, New Taipei City (TW)

(73) Assignee: Hong-Ming Lin, New Taipei City (TW)

(21) Appl. No.: 14/562,893

(22) Filed: Dec. 8, 2014

(30) Foreign Application Priority Data
Apr. 4, 2014 (CN) 201410136155.8

(51) Int. Cl. A01N 63/00; A61L 2/18; A01N 59/16

(52) U.S. Cl. CPC A01N 63/00; A01N 59/16; A61L 2/18

(57) ABSTRACT

The present invention provides a complex which is coordinated by a protease and a nano sliver, wherein the complex having the absorption spectrum shown in FIG. 1 that absorbance at wavelengths 250-300 nm and wavelength 450 nm decreases after 17 hours of coordination. The present invention also provides an antibacterial method comprising applying the complex onto an object. The present invention still provides a method of removing odor produced by microorganisms comprising applying the complex onto an object.

The present application claims priority to Chinese Patent Application No. 201410136155.8 filed on 4 Apr. 2014, incorporated herein by reference in its entirety.

### FIELD OF THE INVENTION

The present invention is related to a complex coordinated by a protease and a nano sliver, and the complex has good antibacterial activity.

### BACKGROUND OF THE INVENTION

Anti-bacteria refer to the process of eliminating bacteria or inhibiting growth and activity of bacteria using chemical and physical methods. Antibacterial materials are the materials capable of inhibiting growth and activity of the bacteria. To improve the health quality of living environment and to inhibit the growth and propagation of detrimental microorganisms, antibacterial materials have been used on various daily commodities. For example, using antibacterial materials in manufacturing refrigerator can extend the expiration date and preserve the foods; using antibacterial materials in manufacturing washing machines can prevent growth of mold and bacteria; using antibacterial materials in producing public equipment can effectively decrease the spreading of the diseases; using antibacterial materials in producing drinking water pipes can prevent attachment and growth of bacteria and algae on the inner pipe wall. Thus, using antibacterial materials on various daily commodities is an effective way to improve health quality and environmental safety.

The acting mechanisms of general antibacterial materials mainly include: (1) interfering synthesis of the cell wall by inhibiting cross-links of polysaccharides and peptide in bacterial cell wall to cause loss of cell wall integrity and the protection mechanism against osmotic pressure; (2) damaging cell membrane by destructing cell membrane to kill bacteria; (3) inhibiting synthesis of proteins by altering or terminating protein synthesis process to kill bacteria; and (4) interfering synthesis of nucleic acids by inhibiting bacterial genetic information including DNA and RNA syntheses in bacteria.

The antibacterial effect of silver was discovered in the early history. In ancient Rome and ancient Persia, human has begun to use silverwares on storage of the liquids to prevent decomposition. In modern era, silver was further used to prevent wounds infection during World War I, and the upscale skin cosmetic manufacturers have been using colloidal silver in reducing the quantity of preservatives in their products in order to prevent preservative-induced allergic reaction of the skin.

Silver itself possesses functions of preventing ulceration and accelerating healing of the wound. The nano silver treated with nanotechnology owing to the dramatic enhancement of its surface area could significantly increase its antibacterial activity. In the mean time, nano silver does not cause harm to human body and retains its antibacterial activity for a long period of time because reactions do not easily deplete its antibacterial ability. The principle of nano silver's anti-bacterial effect is based on the strong binding activities between nano silver particles and cell walls/membranes of the microorganisms. When the positively charged nano silver particles contact with negatively charged microorganism cells, they adhere to each other, leading to directly entering of nano silver particles into the bacteria. Once entering into the bacteria, nano silver particles bind with the sulfhydryl groups (—SH) of the cell membrane proteins, block metabolism and deactivate its activity, thereby inhibiting bacteria growth to generate antibacterial effect. Nano silver is neutral in water, can resist acids, salts and weak bases, and is stable against heat and lights.

Nano silver can ameliorate the adverse effects caused by the conventional use of antibiotics, in addition to the prevention of developing antibiotic-resistance by the bacteria, due to its wide antibacterial spectrum, nano silver possesses antibacterial effect against a large variety of bacteria. Currently nano silver has been widely used in general living environment. Nano silver-related products have been put on the market one after another, including bandages, socks, water dispensers, air conditioners, refrigerators, washing machines and other antibacterial products, indicating a new trend on daily commodities lifted by nano silver.

Protease is one type of enzymes in organisms, capable of digesting proteins. The method of digestion is to break the peptide bonds that connect amino acids to form polypeptide chains. Proteases exist widely inside the internal organs of animals, stems, leaves and fruits of the plants, and microorganisms. Proteases from microorganisms are produced mainly by molds, bacteria, and then yeasts and actinobacteria.

Proteases are molecules produced in organisms and the utilization of proteases for digesting proteins can be used in antibacterial reagents, nevertheless when in high concentration they could cause damages to human body.

### BRIEF DESCRIPTION OF THE DRAWINGS

FIG. 1 shows the absorption spectrum of the complex in the present invention, the “extracts” described herein are protease extracts and are the same hereafter.

FIG. 2 shows the antibacterial effect of the nano silver.

FIG. 3 shows the antibacterial effect of the extracts.

FIG. 4 shows the antibacterial effect of the complex in the present invention.

### SUMMARY OF THE INVENTION

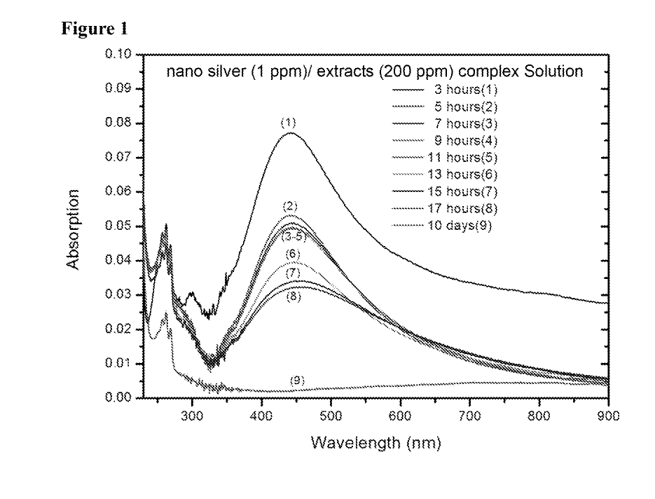

The present invention provides a complex which is coordinated by a protease and a nano sliver, wherein the complex having the absorption spectrum shown in FIG. 1 that absorbance at wavelengths 250-300 nm and wavelength 450 nm decreases after 17 hours of coordination. The present invention also provides an antibacterial method comprising applying the complex onto an object. The present invention still provides a method of removing odor produced by microorganisms comprising applying the complex on an object.

### DETAIL DESCRIPTION OF THE INVENTION

Unless otherwise specified, “a” or “an” means “one or more”.

Due to the effects of the antibacterial concentrations of nano silver and proteases on the human body, the present invention provides a nano silver complex that possesses good antibacterial effect in a low dose.

The “antibacterial activity/effect” described in the present invention includes but is not limited to sterilization, bacteria elimination, disinfection, bacteria inhibition, mold prevention, anti-decomposition, etc.

The “nano silver” described in the present invention refers to the silver particles smaller than 100 nanometer and the “extracts” refer to protease extracts which can be isolated from plants (include but are not limited to pineapple, mango, papaya), animals or microorganisms and also can be prepared by protein recombination technology.

Therefore, the present invention provides a complex which is coordinated by a protease and a nano sliver, wherein the complex having the absorption spectrum shown in FIG. 1 that absorbance at wavelengths 250-300 nm and wavelength 450 nm decreases after 17 hours of coordination.

In a preferred embodiment of the present invention, the nano silver comprises a surfactant and the surfactant includes but is not limited to polysorbate 20 (Tween 20), polyoxyethylene octyl phenyl ether (Triton X-100), polyvinylpyrrolidone (PVP), sodium dodecyl sulfate (SDS) or hexadecyl trimethyl ammonium bromide, cetyltrimethylammonium bromide (CTAB).

In a preferred embodiment of the present invention, concentration of the nano silver is 0.0125-10 ppm. In a more preferred embodiment of the present invention, concentration of the nano silver is 0.1-3 ppm. In another more preferred embodiment of the present invention, concentration of the nano silver is 0.5-2 ppm.

In a preferred embodiment of the present invention, concentration of the protease is 10-500 ppm. In a more preferred embodiment of the present invention, concentration of the protease is 100-400 ppm. In another more preferred embodiment of the present invention, concentration of the protease is 200-300 ppm.

The present invention also provides an antibacterial method comprising applying the complex stated above on an object.

In a preferred embodiment of the present invention, the applying includes but is not limited to spraying, coating, immersing, wiping or rinsing; the object includes but is not limited to public baths, hospitals, kitchens, hotels or food factories and the bacterium includes but is not limited to Klebsiella pneumoniae, Escherichia coli, Candida albicans, Pseudomonas aeruginosa, Staphylococcus aureus or Aspergillus niger.

The present invention also provides a method of removing odor produced by microorganisms comprising applying the complex stated above onto an object. In a preferred embodiment of the present invention, the odor produced by the microorganisms includes but is not limited to odors generated by bacteria or molds or odor of putrefaction.

In a preferred embodiment of the present invention, the applying includes but is not limited to spraying, coating, immersing, wiping or rinsing; the object includes but is not limited to public baths, hospitals, kitchens, hotels or food factories and the bacterium includes but is not limited to Klebsiella pneumoniae, Escherichia coli, Candida albicans, Pseudomonas aeruginosa, Staphylococcus aureus or Aspergillus niger.

### EXAMPLES

The examples below are non-limiting and are merely representative of various aspects and features of the present invention.

Preparation of the Complex

Nano silver solution: the highly concentrated nano silver solution (purchased from Ming Fung Nano-Biotechnology Co., Ltd., New Taipei City, Taiwan) was diluted with deionized water into experimental concentrations; for example, 0.025-3.2 ppm.

Protease extract solution: the original pineapple protease extract solution (purchased from Challenge Bioproducts Co., Ltd., Touliu, Taiwan) was diluted with deionized water into experimental concentrations; for example, 100-400 ppm.

Preparation of nano silver/protease complex solution: the bottles containing different ratios of nano silver and protease were shaken for dispersing for 10 hours at a temperature lower than 50° C. the color of the solution turned from yellow-brown into transparent, yielding stable nano silver/protease complex solution.

The complex in the present invention was coordinated by a protease and a nano silver and had the absorption spectrum shown in FIG. 1, wherein the absorbance at wavelengths 250-300 nm and wavelength 450 nm decreased after 17 hours of coordination.

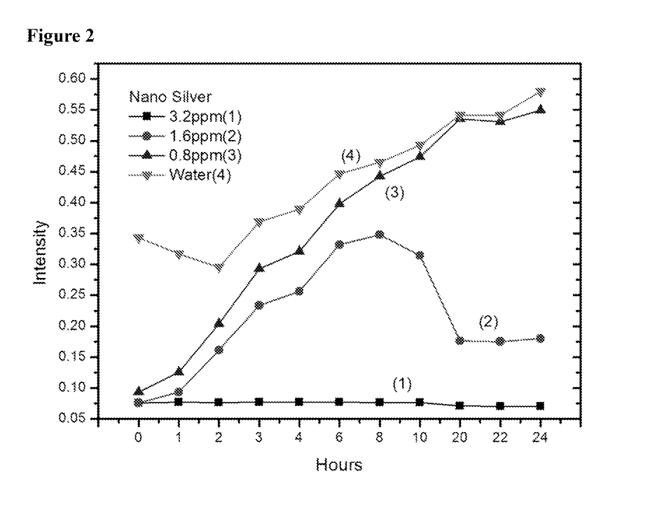

FIG. 2 showed the antibacterial effect of the nano silver. When the concentration of the nano silver reached 1.6 ppm, significant antibacterial effect was shown after 10 hours of reaction; when the concentration of the nano silver reached 3.2 ppm, the tested bacteria almost stopped growing. Nevertheless, when the concentration of the nano silver was 0.8 ppm, the antibacterial effect of the nano silver was almost absent.

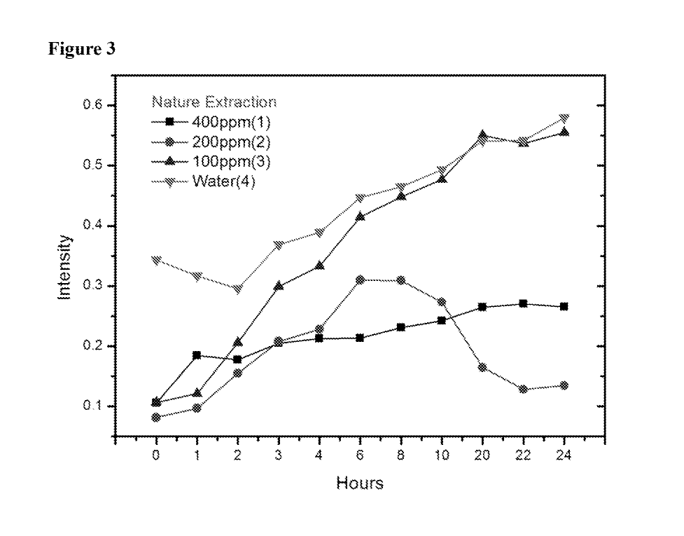

FIG. 3 showed the antibacterial effect of the extracts. When the concentration of the protease extract was 200 ppm, significant antibacterial effect was present after 8 hours of reaction. Therefore, this concentration was selected for the subsequent experiments.

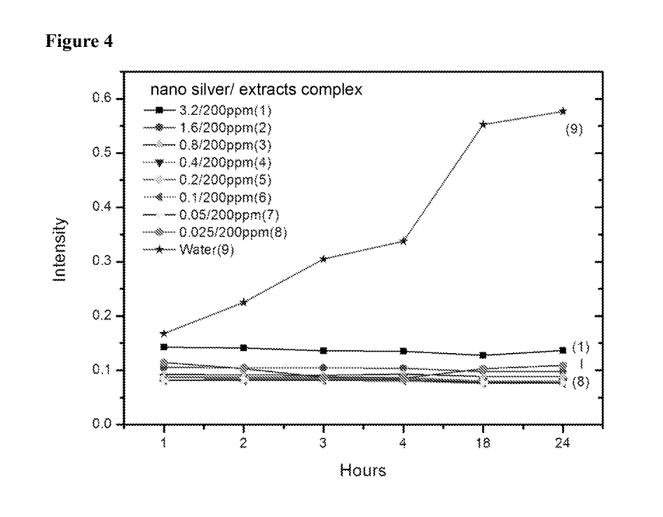

FIG. 4 showed the antibacterial effect of the complex in the present invention. When the nano silver and the protease formed the complex, the complex possessed effective antibacterial function even with a low concentration of the nano silver at 0.025 ppm and the tested bacteria almost stopped growing.

Antibacterial Test of the Complex

The complex with 1 ppm nano silver coordinated with 200 ppm protease extract was used for antibacterial test according to the U.S. Pharmacopeia microbial antimicrobial preservatives effectiveness test (51). The complex and the bacteria suspension were mixed at 20 ml:0.2 ml respectively and then tested, the results are shown in the following table:

| | | Reacting time | | |
|---|---|---|---|---|
| | | | 1-hour | Anti- |
| | | Initial | post-reaction | bacte- |
| | | bacteria | bacteria | rial |
| Bacteria strain | | quantity | quantity | rate (%) |
| Staphylococcus aureus | (CFU/ml) | 1.3 × 10⁵ | <1 | 99.999% |
| ATCC6538 | | | | |
| Escherichia coli | (CFU/ml) | 4.0 × 10⁵ | <1 | 99.999% |
| ATCC8739 | | | | |
| Pseudomonas | (CFU/ml) | 9.0 × 10⁵ | <1 | 99.999% |
| aeruginosa | | | | |
| ATCC9027 | | | | |
| Klebsiella pneumoniae | (CFU/ml) | 1.6 × 10⁵ | <1 | 99.999% |
| ATCC4352 | | | | |
| Candida albicans | (CFU/ml) | 1.3 × 10⁵ | <1 | 99.999% |
| ATCC 10231 | | | | |
| Aspergillus niger | (CFU/ml) | 1.1 × 10⁵ | 3.0 × 10¹ | 99.9% |
| ATCC16404 | | | | |

Antibacterial Time Effect of the Complex

The complex with 1 ppm nano silver coordinated with 200 ppm protease extract was used for antibacterial test according to U.S. Pharmacopeia microbial antimicrobial preservatives effectiveness test (51). The complex and the bacteria suspension were mixed at 20 ml:0.2 ml respectively and then after 8 and 24 hours of antibacterial test, the bacteria quantity was tested, the results are shown in the following table:

| | | Reacting time | | |
|---|---|---|---|---|
| | | | 8 hours | Anti- |
| | | Initial | post-reaction | bacte- |
| | | bacteria | bacteria | rial |
| Bacteria strain | | quantity | quantity | rate (%) |
| Staphylococcus aureus | (CFU/ml) | 2.8 × 10⁵ | <1 | 99.99% |
| ATCC6538 | | | | |
| Escherichia coli | (CFU/ml) | 3.1 × 10⁵ | <1 | 99.99% |
| ATCC8739 | | | | |
| | | | 24 hours | Anti- |
| | | Initial | post-reaction | bacte- |
| | | bacteria | bacteria | rial |
| Bacteria strain | | quantity | quantity | rate (%) |
| Staphylococcus aureus | (CFU/ml) | 2.8 × 10⁵ | <1 | 99.99% |
| ATCC6538 | | | | |
| Escherichia coli | (CFU/ml) | 3.1 × 10⁵ | <1 | 99.99% |
| ATCC8739 | | | | |

According to the embodiments described above, when the nano silver and the protease were formed into a complex, its antibacterial effect was much greater than the combined effects of each individually. This complex greatly reduces the quantity of antibacterial reagent used as well as its impact on the environment and organisms. The complex also showed long term antibacterial effects.

Although the present invention is disclosed as the embodiments described above, they are not used to limit the present invention, some variation and modification can be made by any person with ordinary skill in the art without departing from the scope and spirit of the present invention. Therefore, the scope of the present invention is defined by the claims appended within the present specification.

What is claimed is:

1. A complex which is coordinated by a protease and a nano sliver, wherein the complex having the absorption spectrum shown in FIG. 1 that absorbance at wavelengths 250-300 nm and wavelength 450 nm decreases after 17 hours of coordination.

2. The complex of claim 1, wherein concentration of the nano silver is 0.0125-10 ppm.

3. The complex of claim 1, wherein concentration of the protease is 10-500 ppm.

4. The complex of claim 1, wherein the nano silver comprises a surfactant.

5. The complex of claim 4, wherein the surfactant is polysorbate 20 (Tween 20), polyoxyethylene octyl phenyl ether (Triton X-100), polyvinylpyrrolidone (PVP), sodium dodecyl sulfate (SDS) or hexadecyl trimethyl ammonium bromide, cetyltrimethylammonium bromide (CTAB).

6. An antibacterial method comprising applying the complex of claim 1 onto an object.

7. The method of claim 6, wherein the applying is spraying, coating, immersing, wiping or rinsing.

8. The method of claim 6, wherein the object is selected from public baths, hospitals, kitchens, hotels or food factories.

9. The method of claim 6, wherein the bacterium is Klebsiella pneumoniae, Escherichia coli, Candida albicans, Pseudomonas aeruginosa, Staphylococcus aureus or Aspergillus niger.

10. A method of removing odor produced by microorganisms comprising applying the complex of claim 1 onto an object.

11. The method of claim 10, wherein the odor produced by the microorganisms comprises odors generated by bacteria or molds or odor of putrefaction.

12. The method of claim 10, wherein the applying is spraying, coating, immersing, wiping or rinsing.

13. The method of claim 10, wherein the object is selected from public baths, hospitals, kitchens, hotels or food factories.
